(19) United States
(12) Patent Application Publication
(10) Pub. No.: US 2008/0114305 A1
(43) Pub. Date: May 15, 2008

(54) Botox® Injector

(76) Inventor: Scott J. Gerondale, Newbury Park, CA (US)

Correspondence Address: ALLERGAN, INC., 2525 DUPONT DRIVE, T2-7H, IRVINE, CA, 92612-1599, US

(21) Appl. No.: 11/572,096

(22) PCT Filed: Aug. 8, 2005
(86) PCT No.: PCT/US05/28525

§ 371 Date: Sep. 25, 2007

Related U.S. Application Data
(60) Provisional application No. 60/600,555, filed on Aug. 10, 2004.
(60) Provisional application No. 60/600,552, filed on Aug. 10, 2004.
(60) Provisional application No. 60/601,814, filed on Aug. 16, 2004.

(51) Int. Cl. A61M 5/28; A61M 5/315

(52) U.S. Cl. 604/207; 604/239

(57) ABSTRACT

An injection including a syringe having a barrel for containing a medicament, a shell for receiving the barrel with a needle projecting therefrom, a drive mechanism for moving a plunger disposed within the barrel, and a manually operative mechanism for moving the plunger in a stepwise manner to eject doses of the medicament from the barrel through the needle.

### CROSS REFERENCE TO RELATED APPLICATIONS

This is a national stage application under 35 U.S.C. § 371 of PCT application PCT/US2005/028525, filed on Aug. 8, 2005, which claims the benefit of Provisional Application Number 60/600,555, 60/600,552, filed on Aug. 10, 2006 and 60/601,814 filed on Aug. 16, 2004.

The present invention is generally directed to a multiple dose injection device and more particularly to a multiple dose injector suitable for precise placement of desired amounts of BOTOX® to specific muscle tissue.

Current procedures for injection of BOTOX® utilize a syringe and the injection volume is controlled by a users' ability to stop on graduations indicated on a side of the syringe.

The present invention provides for better control of the BOTOX® injection.

### SUMMARY OF THE INVENTION

One embodiment of the present invention includes a controlled volume injector/aspiration device that generally includes a syringe having a barrel or body for containing a medicament, a needle, and a piston slidably disposed in the body.

A shell is provided for receiving the syringe body with the needle projecting from the shell and a driver is disposed within the shell for moving the piston.

A drive screw having a first and a second end is disposed in the shell and a drive net for releasably engaging the drive screw proximate the first end is provided for causing movement of the driver and piston upon rotation of the drive screw when engaged by the drive nut.

Upon disengagement of the drive nut reverse movement of the driver and piston is enabled thus providing aspiration through the needle.

A pinion is disposed on the drive screw second end for rotation therewith and a drive pawl causes stepwise rotation of the pinion and drive screw.

A manually operated button is disposed in an operative relationship with the pinion and pawl for rotating the drive screw and moving the driver and piston in the stepwise forward direction causing the piston to eject discrete doses of medicament from the syringe body through the needle. Preferably, the medicament is botulinum toxin.

In another embodiment of the present invention a syringe form injector generally includes a syringe having a barrel for containing a medicament, a needle, and a plunger slidably disposed in the barrel. A shell is provided for receiving the syringe barrel with the needle projecting from one end of the shell and a toothed rack is disposed within the shell for moving the plunger.

A drive pawl is provided for causing stepwise movement of the toothed rack.

A manually operated button is disposed at another end of the shell and further disposed in an operative relationship with the pawl for moving the rack and plunger in a stepwise forward direction causing the plunger to eject discrete doses of medicament from the syringe barrel through the needle.

A draw up handle is disposed in an operative relationship with the rack for withdrawing the plunger from the syringe barrel to aspirate medicament or other fluid into the barrel.

More particularly, the syringe form injector in accordance with the present invention may include a volume of medicament, preferably botulinum toxin.

In yet another embodiment of the present invention an escapement needle injector generally includes a housing and a syringe having a barrel and a needle disposed within the housing with the needle protruding from the housing.

A double wall plunger is provided having a piston slidably disposed within the barrel and an outer wall slidably disposed exterior to the barrel. The outer wall includes a linear array of teeth therealong.

A toothed drive wheel is provided for engaging the linear array of teeth and moving the piston into the syringe barrel. In addition, a toothed drive wheel advancing gear is fixed to the drive wheel for rotation therewith.

A pawl is provided for engaging the drive gear and causing stepwise rotation thereof, such rotation being translated to the toothed drive wheel for stepwise advancing of the piston into the barrel.

A trigger is provided, coupled to the housing and disposed in an operative relationship with the pawl for rotating the drive gear and drive wheel in a stepwise manner causing the piston to eject discrete doses of medicament from the syringe barrel through the needle.

A draw up handle is fixed to the plunger outer wall for withdrawing the piston from the syringe barrel to either aspirate fluids or introduce medicament into the barrel. Alternatively, medicament may be initially provided within the barrel.

### BRIEF DESCRIPTION OF THE DRAWINGS

The present invention may be more clearly understood with reference to the following detailed description in conjunction with the appended drawings of which:

FIG. 1 is a perspective view of a controlled volume injection/aspiration device in accordance with the present invention generally showing a shell and a manually operated button for injecting discrete doses of medicament through a needle projecting from the shell;

FIG. 2 is a cross sectional view of the device shown in FIG. 1 illustrating a shell, a driver, a drive screw, a drive nut, a pinion, drive pawl, piston, and a manually operated button;

FIG. 3 is a view similar to that shown in FIG. 2 with the piston moved forwardly within the syringe body;

FIG. 4 is a cross sectional view taken along the line 4-4 of FIG. 2;

FIG. 5 is a view similar to FIG. 4 illustrating disengagement of a drive nut and lever;

FIG. 6 is a cross sectional view illustrating operation of the drive pawl for stepwise rotation of the pinion and drive screw;

FIG. 7 is a perspective view of a syringe form injector in accordance with the present invention generally showing a shell with a syringe needle protruding therefrom, a manually operated button and finger supports;

FIG. 8 is a perspective view of a syringe and operating mechanism disposed within the shell shown in FIG. 7;

FIG. 9 is another perspective view of the syringe in operating mechanism shown in FIG. 8;

FIG. 10 is a cross sectional view of the syringe form injector in accordance with the present invention more particularly showing a syringe barrel and operating mechanism including a toothed rack, pawl button, and a draw up handle;

FIG. 11 is a perspective view of an escapement needle ejector in accordance with the present invention generally showing a housing, a syringe needle protruding therefrom, a grip handle, and trigger;

FIG. 12 is a perspective view of the operating mechanism disposed within the housing shown in FIG. 11 generally showing a syringe having a barrel along with a double wall plunger, including an outer wall with a linear array of teeth therealong, a toothed drive wheel, a toothed drive wheel advancing gear along with a trigger;

FIG. 13 is a view similar to that shown in FIG. 12 with the trigger removed to more clearly show the drive wheel advancing gear, an advancing pawl and a trigger spring; and

FIG. 14 is a perspective view similar to that shown in FIG. 13 showing an opposite side of the drive wheel with the trigger operating spring connected thereto.

### DETAILED DESCRIPTION

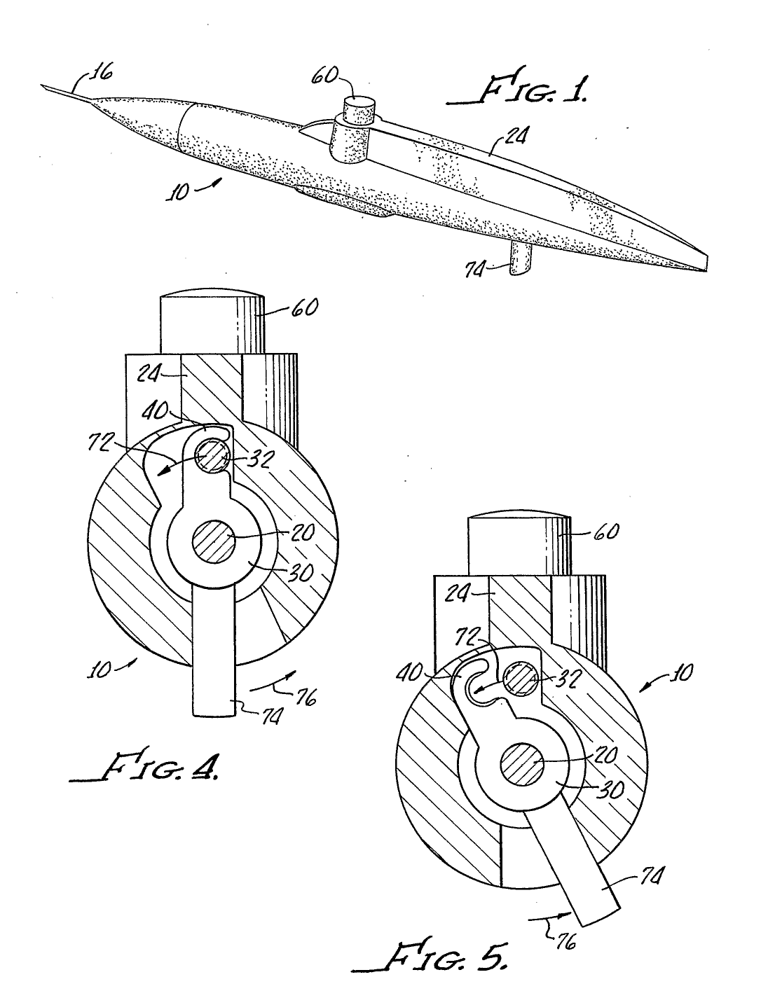

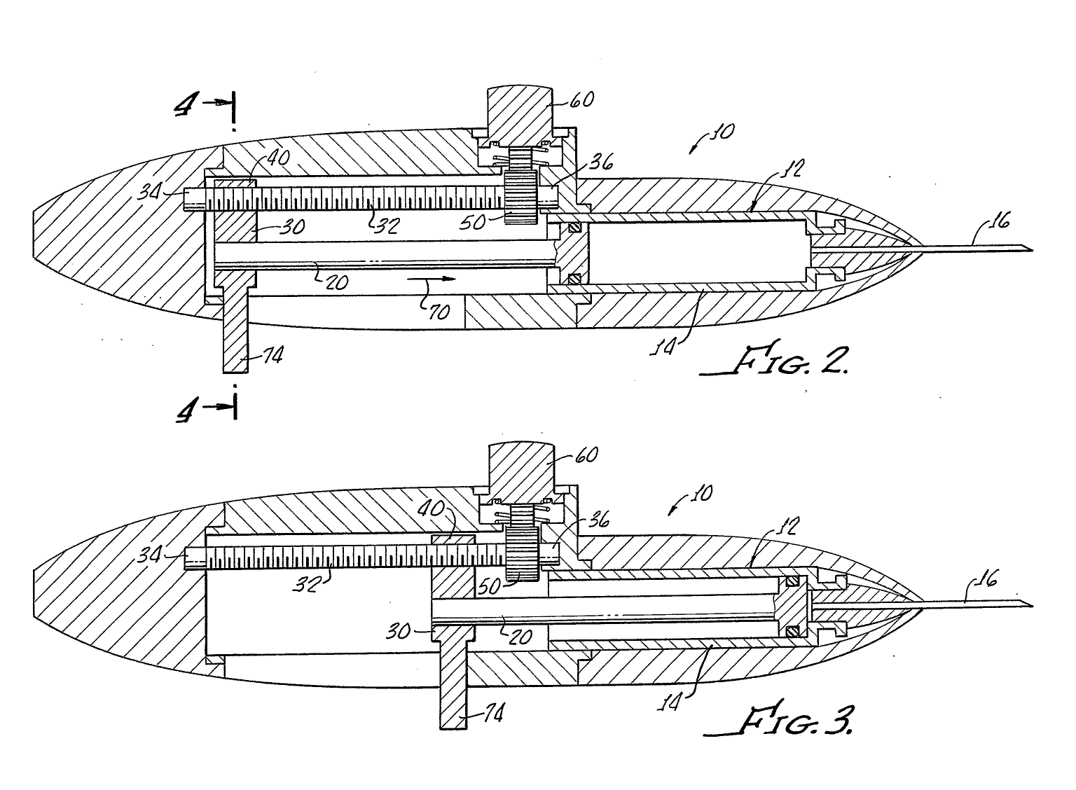

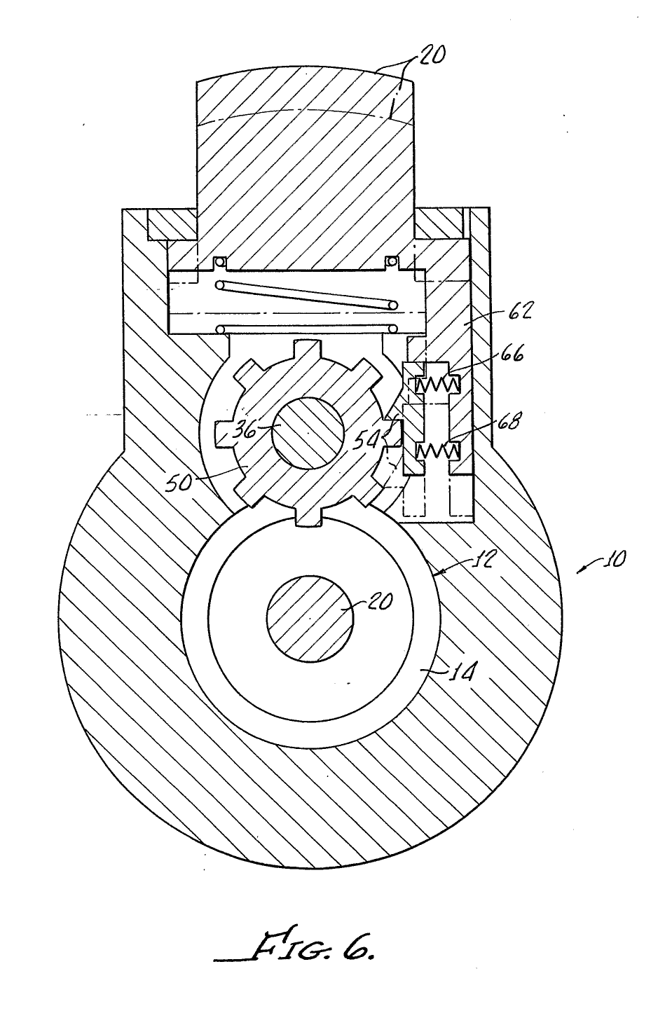

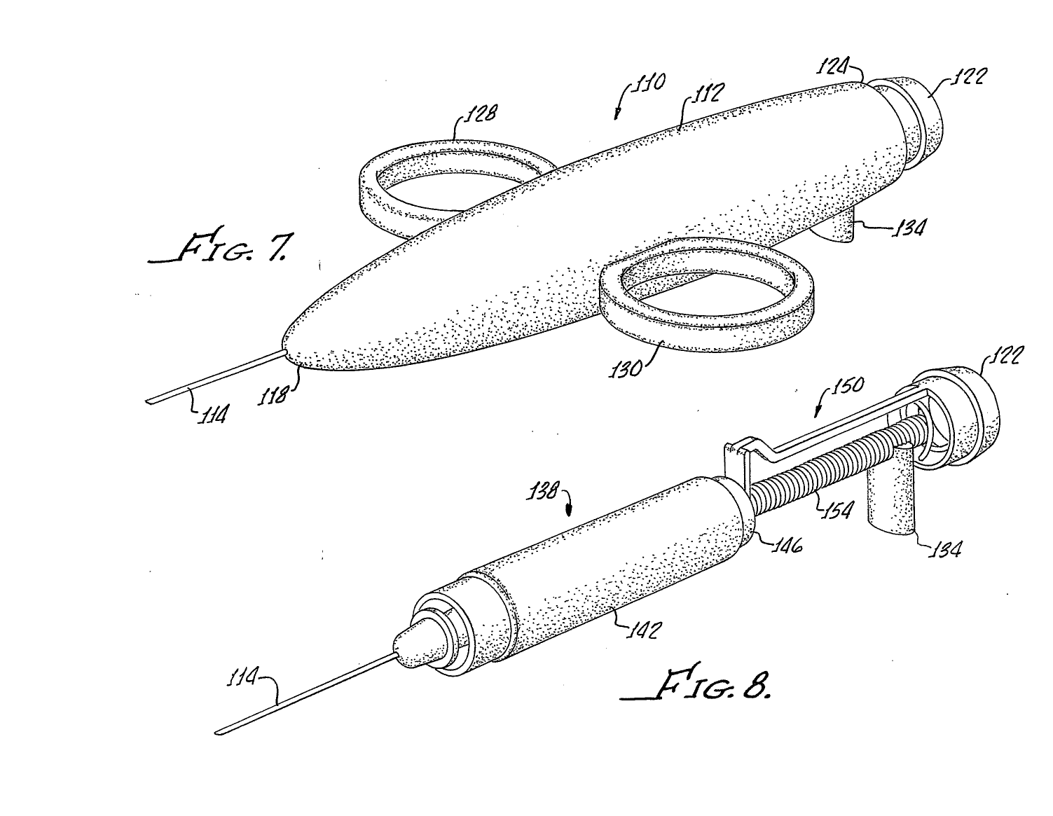

With reference to FIGS. 1-6 a controlled volume injector/aspiration device 10 in accordance with the present invention generally includes a syringe 12 having a barrel or body 14, a needle 16, and a piston 20 slidably disposed in a body 14.

A shell 24 is provided for receiving the syringe body 14 with the needle 16 projecting from the shell 24.

A driver 30 is disposed within the shell 24 for moving the piston 20 by way of a drive screw 32 having a first end 34 and a second end 36.

A drive nut 40 is provided for releasably engaging the drive screw 32 proximate the first end 34. This is facilitated by an open side 44 of the nut 40.

When the drive nut 40 is engaged with the drive screw 32, rotation of the drive screw causes movement of the driver and piston.

When the drive nut 40 is disengaged from the drive screw 32, reverse movement of the driver and piston is enabled, thus enabling aspiration through the needle 16.

A pinion is disposed on the second end 36 of the drive screw 32 for rotation therewith and a drive pawl 54 is provided for stepwise rotation of the pinion 50 and drive screw 32 through the use of a depressible button 60 and linkage 62. Springs 66, 68 enable engagement and disengagement of the drive pawl 54 with the pinion 50 in a conventional manner.

Thus, when the manually operated button 60 is depressed, the pawl 54 rotates the drive screw 32 moving the driver 30 and piston 20 in a stepwise forward direction indicated by the arrow 70 to eject discrete doses of medicament, such as BOTOX®, from the syringe body 14 through the needle 16.

When the drive nut 60 is disengaged from the drive screw by rotation of the nut 40, as shown by the arrow 72, by lever 74, reverse movement of the piston 20 in the direction of arrow 76 is also effected through the lever 74.

With reference now to FIG. 7, there is shown a syringe form injector 110 in accordance with the present invention generally showing a shell 112 with a syringe needle 114 projecting from one end 118 of the shell and a manually operated button 122 disposed at another end 124 of the shell 112. A pair of finger rings 128, 130 preferably molded with the shell on opposite sides thereof are provided for facilitating operation of the injector 110. An operation forefingers (not shown) are placed through the finger rings 128, 130 and a thumb, not shown, is utilized to manually press the button 122. As will be shown and described hereinafter, a draw up handle 134 is provided for aspirating medicament or fluid through the needle 114.

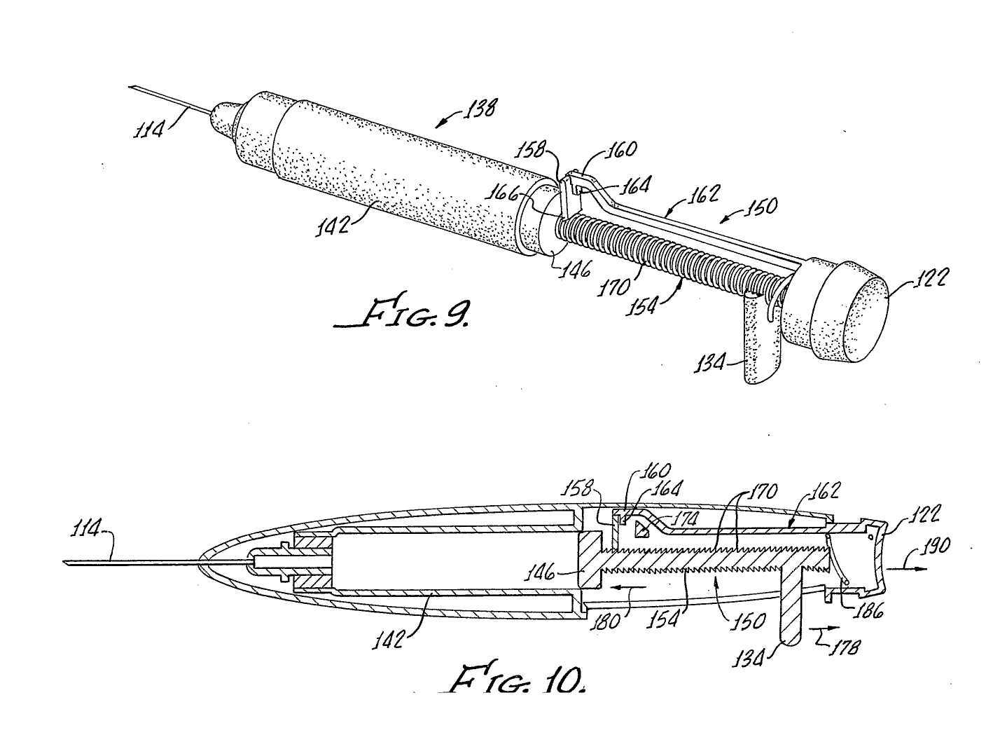

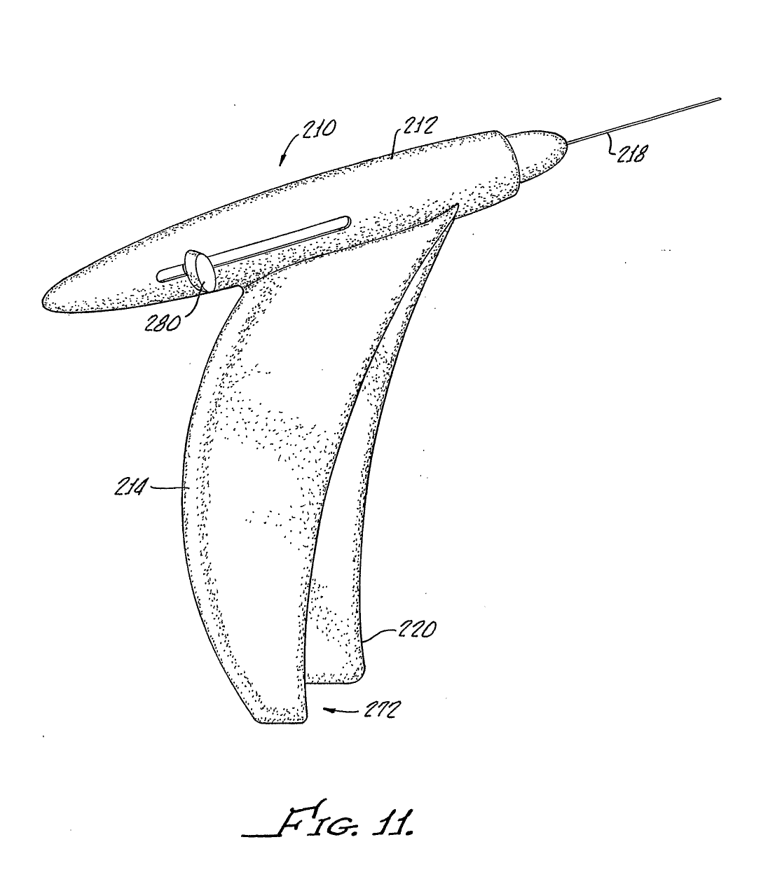

With reference to FIGS. 8-10, a syringe 138 includes the needle 134 and a syringe barrel 142 and a plunger 146 slidably disposed within the barrel 142.

An operating mechanism 150 includes a toothed rack 154, which may be of circular cross section, disposed within the shell 112 for moving the plunger 146.

A drive pawl is pivotally attached through one end of an actuator, or advancer, 162 for intermittently engaging the rack 154. A stop prevents rearward movement of the pawl 158 and a beveled end 166 of the pawl 158 engages teeth 170 on the rack 154.

As shown in FIG. 10, a disengaging cam 174 may be provided for lifting the actuator end 160 and pawl 158 to enable aspiration through the needle by the draw up handle which is attached to the rack 154 in a reverse direction, as indicated by the arrow 178.

The button 122 is interconnected with the advancer 162 causing it to be in an operative relationship with the pawl 158 for moving the rack 154 and a plunger 146 in a stepwise forward direction indicated by an arrow 180 causing the plunger 146 to eject discrete doses of medicament from the syringe barrel 142 through the needle 134.

A return spring 186 is disposed in an operative relationship with the button 122 for outward movement of the button 122 indicated by the arrow 190 after depression of the button 122 causing the forward stepwise motion of the rack 154 and plunger 146. The pivotal mounting of the pawl enables the pawl 158 to ride over the teeth 154 as the button is moved outwardly by the spring 186. This initializes the operating mechanism 154 for sequential ejection of medicament through another depression of the button 122.

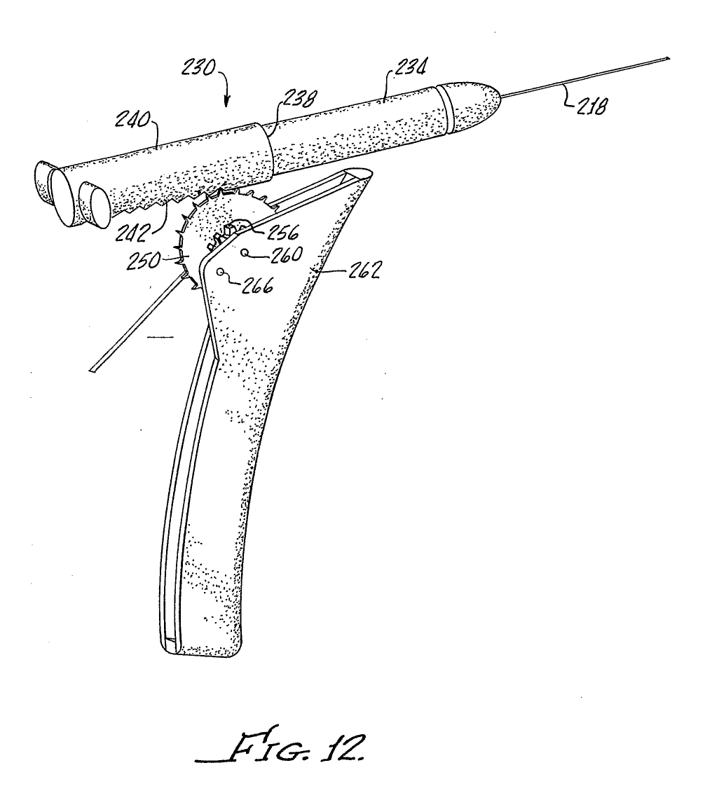

With further reference to FIG. 11, there is shown an escapement needle ejector 210 in accordance with the present invention generally showing a housing 212 and a depending handle 214 connected thereto. The housing 212 may be transparent. Also shown is a needle 218 protruding from the housing 212 along with a squeezable trigger 220.

With reference to FIG. 12, there is shown an operating mechanism 230 including a syringe barrel 234 and a piston 238 slidably disposed within the barrel 234 and having an outer wall 240 slidably disposed exterior to the barrel 234 and including a linear array of teeth 242 therealong.

A hub with a luer lock is provided for securing the needle 218 to the syringe barrel 234.

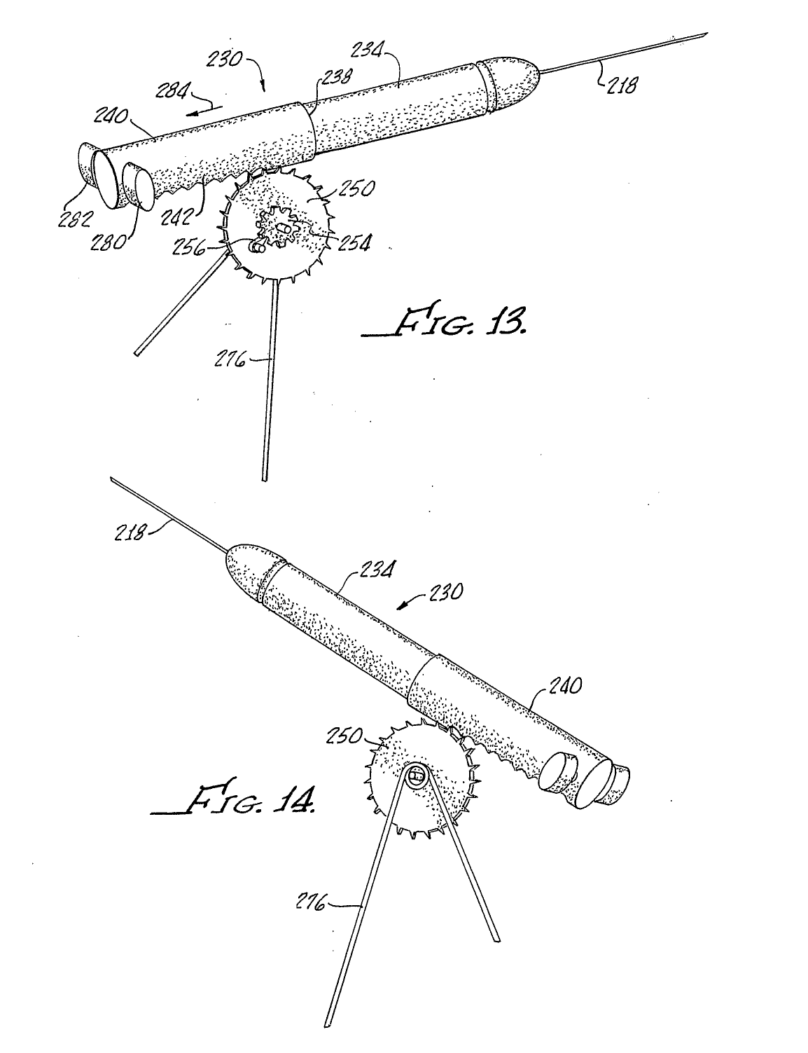

As shown in FIGS. 12-14, a toothed drive wheel 250 engages the linear array of teeth 242 on the plunger outer wall 240 for moving the piston 238 in a stepwise manner for ejecting discrete doses of medicament disposed within the barrel 234 through the needle 218.

A toothed drive wheel advancing gear 254 is fixed to the drive wheel for rotation therewith and a pawl 256 is provided for engaging the gear 254 in a conventional manner for causing stepwise rotation thereof, the stepwise rotation being translated to the drive wheel 250, which in turn engages the teeth 242 for stepwise advancement of the plunger 240 and piston 238 along and within the syringe barrel 234.

As shown in FIGS. 12-14, the drive wheel 250 and gear 254 are mounted for rotation by a pin 260 extending through a trigger 262 and the pawl 256 is pin 266 mounted to the trigger 262. Movement of the trigger 262, as indicated by the arrow 272, by manual squeezing thereof engages the pawl 256 with the advancing gear 254, thus causing stepwise rotation thereof.

Accordingly, the trigger 262 is disposed in an operative relationship with the pawl 256 for rotating the drive gear 254 and drive wheel 250 in a stepwise manner causing the piston 238 to eject a discrete dose of medicament from the syringe barrel 234 through the needle 218. A spring 276 causes return of the trigger 220 to an initial position and resets the pawl 256 for repeated engagement with the advancing gear 254.

Draw up handles 280, 282 fixed to the plunger outer wall 240 are provided for moving the piston end plunger wall in reverse direction indicated by the arrow 284 in order to aspirate fluid into the syringe barrel or draw medicament into the syringe barrel if not initially provided therein.

The gun-shaped injector 210 is designed to administer one dose with every squeeze of the trigger 220. The advantages of this device 210 are that the hand of a physician (not shown) is moved away from the centerline of the injection enhancing visibility of the injection site. The internal markings of the barrel 234 are easily visualized through the transparent housing or shell 212. Superior mechanical advantage provides smooth and controlled injection pressure. Drugs may be drawn up into the device by using two lateral handling 280, 282 on the sides of the barrel. With thumb on the proximal end of device, forefinger and second finger are employed to slide handling 280, 282 to the rear, moving the piston 238 in the barrel 234.

Although there has been hereinabove described specific BOTOX® injectors in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. An injection comprising:
a syringe having a barrel for containing a medicament;
a shell for receiving the barrel with a needle projecting therefrom;
a drive mechanism for moving a plunger disposed within said barrel;
a manually operative mechanism for moving said plunger in a stepwise manner to eject doses of the medicament from said barrel through said needle.

2. A controlled volume injection/aspiration device comprising:
a syringe having a body for containing a medicament, a needle and a piston slidably disposed in said body;
a shell for receiving the syringe body with said needle projecting from said shell;
a driver disposed within said shell for moving said piston;
a drive screw, having a first end and a second end, disposed in said shell;
a drive nut for releasably engaging said drive screw proximate the first end, the drive nut when engaged with said drive screw causing movement of said driver and piston upon rotation of said drive screw, the drive nut when disengaged from said drive screw enabling reverse movement of said driver and piston;
a pinion disposed on the drive screw second end for rotation therewith;
a drive pawl for stepwise rotation of said pinion and drive screw;
a manually operated button disposed in an operative relationship with said pinion and pawl for rotating said drive screw and moving said driver and piston in a stepwise forward direction causing said piston to eject discrete doses of medicament from said syringe body through said needle; and
a lever disposed in an operative relationship with said drive nut for engaging and disengaging said drive nut with end and apart for said drive screw.

3. A controlled volume injection/aspiration device comprising:
a syringe having a body, a needle and a piston slidably disposed in said body;
a medicament disposed in said body;
a shell for receiving the syringe body with said needle projecting from said shell;
a driver disposed within said shell for moving said piston;
a drive screw, having a first end and a second end, disposed in said shell;
a drive nut for releasably engaging said drive screw proximate the first end, the drive nut when engaged with said drive screw causing movement of said driver and piston upon rotation of said drive screw, the drive nut when disengaged from said drive screw enabling reverse movement of said driver and piston;
a pinion disposed on the drive screw second end for rotation therewith;
a drive pawl for stepwise rotation of said pinion and drive screw;
a manually operated button disposed in an operative relationship with said pinion and pawl for rotating said drive screw and moving said driver and piston in a stepwise forward direction causing said piston to eject discrete doses of medicament from said syringe body through said needle; and
a lever disposed in an operative relationship with said drive nut for engaging and disengaging said drive nut with end and apart for said drive screw.

4. The device according to claim 3 wherein said medicament comprises botulinum toxin.

5. A syringe form injector comprising:
a syringe having a barrel for containing a medicament, a needle and a plunger slidably disposed in said barrel;
a shell for receiving the syringe barrel with said needle projecting from one end of said shell;
a toothed rack disposed within said shell for moving said plunger;
a drive pawl for causing stepwise movement of said toothed rack;
a manually operated button disposed at another end of said shell and in an operative relationship with said pawl for moving said plunger in a stepwise forward direction causing said plunger to eject discrete doses of the medicament from said syringe barrel through said needle; and
a draw up handle disposed in an operative relationship with said each for withdrawing said plunger from the syringe barrel.

6. A syringe form injector comprising:
a syringe having a barrel, a needle and a piston slidably disposed in said barrel;
a medicament disposed in said barrel;
a shell for receiving the syringe barrel with said needle projecting from one end of said shell;
a toothed rack disposed within said shell for moving said plunger;
a drive pawl for causing stepwise movement of said toothed inch;
a manually operated button disposed in an operative relationship with said pawl for moving said rack and plunger in a stepwise forward direction causing said plunger to eject discrete doses of medicament from said syringe barrel through said needle; and
a draw up handle disposed in an operative relationship with said rack for withdrawing said plunger from the syringe barrel.

7. The device according to claim 6 wherein said medicament comprises botulinum toxin.

8. An escapement needle injector comprising:
a housing;
a syringe having a barrel and a needle disposed within said housing with the needle protruding therefrom;
a plunger having a piston slidably disposed within said barrel and an outer wall slidably disposed entering to said barrel, said outer wall including a linear array of teeth therealong;
a toothed drive wheel for engaging the linear array of teeth and moving said piston into the syringe barrel;
a toothed drive wheel advancing gear fixed to said drive wheel for rotation therewith;
a pawl for engaging said drive gear and causing stepwise rotation thereof, the stepwise rotation being translating to said toothed drive wheel for stepwise advance of said piston into said barrel;
a trigger disposed in an operative relationship with said panel for rotating the drive gear and drive wheel in a stepwise manner carrying said piston to eject discrete doses of medicament from the syringe barrel through said needle; and
draw up handle fixed to the plunger outer wall for withdrawing said piston from the syringe barrel.

9. An escapement needle injector comprising:
a housing;
a syringe having a barrel and a needle disposed within said housing with the needle protruding therefrom;
a medicament disposed in said barrel;
a plunger having a piston slidably disposed within said barrel and an outer wall slidably disposed entering to said barrel, said outer wall including a linear array of teeth therealong;
a toothed drive wheel for engaging the linear array of teeth and moving said piston into the syringe barrel;
a toothed drive wheel advancing gear fixed to said drive wheel for rotation therewith;
a panel for engaging said drive gear and causing stepwise rotation thereof, the stepwise rotation being translating to said toothed drive wheel for stepwise advance of said piston into said barrel;
a trigger disposed in an operative relationship with said panel for rotating the drive gear and drive wheel in a stepwise manner carrying said piston to eject discrete doses of medicament from the syringe barrel through said needle; and
draw up handle fixed to the plunger outer wall for withdrawing said piston from the syringe barrel.

10. The injection according to claim 9 wherein said medicament comprises BOTOX®.
